(19) United States
(12) Patent Application Publication
(10) Pub. No.: US 2017/0095375 A1
(43) Pub. Date: Apr. 6, 2017

(54) SANITARY ABSORBING CONSTRUCTION

(71) Applicant: Fan WU, Poggio A Caiano (PO) (IT)

(72) Inventor: Marco BENEDETTI, Bolzano (BZ) (IT)

(21) Appl. No.: 15/128,077

(22) PCT Filed: Mar. 20, 2015
(86) PCT No.: PCT/IB2015/000383

§ 371 Date: Sep. 21, 2016

(30) Foreign Application Priority Data
Mar. 21, 2014 (IT) MI2014A000485

(51) Int. Cl. A61F 13/15

(52) U.S. Cl. CPC A61F 13/15252; A61F 2013/1526; A61F 2013/51019

(57) ABSTRACT

A sanitary absorbing construction to be used by children and infants to hold and restrain liquid and solid dejections, which article is conventionally called napkin, of a single-use or disposable kind, made by combining different raw synthetic or natural materials, comprising apple waste materials, being industrially transformed into composite sub-layers which are assembled together to form articles having different shapes depending on technical or aesthetical requirements, and being biodegradable either before or after their use, when released into the environment, and being compostable or able to be transformed to into organic amending materials, when properly collected and separated or mixed with other organic domestic waste materials and then let into the compost cycle, per se or together with organic waste materials either from foodstuff or not.

### BACKGROUND OF THE INVENTION

The present invention relates to a sanitary absorbing or napkin construction, particularly designed for meeting child physiologic needs.

In particular, the present invention relates to an absorbing article of manufacture to be used by children and infants to hold and restrain human liquid and solid dejections, which article is conventionally called absorbing construction or napkin, of a single-use or disposable kind.

At present, the above articles of manufactures constitute, jointly to the organic waste materials (dejections) which are absorbed or collected thereby, a very large urban waste mass, which, according to statistics studies, varies from 5 to 18% of the urban waste material mass which is produced per day in western civilizations and which articles are considered, in the worldwide culture or economy, indispensable articles in the child or infant sanitary or hygienic field.

The above problem is further worsened by the fact that commercially available absorbing articles of manufacture, in most cases, comprise non-biodegradable plastics materials or, most importantly, non-compostable structural materials.

In fact, prior absorbing articles or napkins are generally constituted by a plurality of overlapping layers, so arranged as to form a filtering sandwich construction, allowing, on a side, a passage of liquids therethrough, while providing, on the other side, a liquid barrier.

Moreover, prior absorbing constructions usually comprise large amounts of grains of synthetic superabsorbing polymeric materials.

In addition, prior absorbing constructions comprise closure systems and other elements for allowing a proper operation, which elements are also made of synthetic plastics materials.

Said prior absorbing constructions further comprise chemical aids and in some cases creams for improving their performance, for example for reducing overheating skin irritations and skin abrasions.

Sanitary napkins for children suffering from incontinence comprise a multi-layer construction, in which each construction layer corresponds to a specific operating function, the combination of which allows liquids to pass through while restraining organic waste materials such as urine and restraining or hindering ejections of solid organic waste or fecal materials.

A sanitary napkin is daily worn and replaced, on average, several times per day.

In industrialized countries, the average child napkin wearing period is up to about 30 months.

Commercially available napkin products have a same construction both for winter and summer periods, notwithstanding the great environment temperature changes; moreover, no structural difference is provided for an absorbing product to be used during day time and night time, if said napkin is worn for more than day time.

Accordingly, such a napkin should be designed based on designing criteria such as, at first, a high comfort; in other words, it should be able of reducing to a minimum any motion difficulties, because of its internal size and weight much larger than those of a child textile or fabric underwear, and also compared to the weight of the child wearing it.

In any case, it should not hinder a proper child physical and psychical development in his/her growth period, in particular a child autonomous deambulation.

A further important criterion to be met, is that the napkin be free from contraindications for the health of the child wearing it, that is it should not cause skin irritations or abrasions and allergic reactions even for children suffering from dermatitis, psoriasis and other skin diseases, or in a stressed or hardened skin condition.

Furthermore, a sanitary napkin should be structurally simply and easily replaceable.

Accordingly, it is necessary to provide single-use or disposable absorbing articles of manufacture which, at the end of their useful life, may be transformed, in a truly natural manner, into novel environmentally useful organic substances, for example for manuring agricultural soils polluted and impoverished by intensive agriculture methods not using natural fertilizing materials.

Another very important requirement to be met is that of making single-use or disposable absorbing articles of manufacture, which, under proper conditions and by dedicated processing systems, can provide gas materials for generating clean energy according to known techniques, for example by a controlled natural fermentation and related anaerobic processes.

Another very important requirement to be met is to provide single-use or disposable absorbing articles of manufacture not aggressive for the user's skin and without contraindications, even after a long time contact with the user's body.

Document GB 2 492 171 A discloses a sanitary article comprising a biodegradable plastics material including spores of a Bacillus species; however, the article of manufacture disclosed by this document is not a compostable article.

Document U.S. Pat. No. 5,190,533 A discloses a biodegradable absorbing construction to be used in napkins and hygienic absorbing structures, which, however, is only suitable to be discharged into a toilet and is not a compostable one.

Document U.S. Pat. No. 6,143,947 A discloses biodegradable and compostable sanitary articles which, however, use several chemical compounds, in particular hydroxyalkanoate; 3-hydroxybutirrate and 3-hydroxyhexanoate.

Document U.S. Pat. No. 5,300,358 A discloses biodegradable and compostable absorbing constructions to be preferably discharged into hygienic bowls, and comprising a non-rigid absorbing degradable fibrous core and a cold water soluble rear or back sheet element but of a waterproof nature and having an outer discontinuous water-repellent surface comprising a substantially discontinuous pattern water-repellent surface material covering about 80% of the rear or back sheet surface and having a critical surface tension lower than 54 dynes/cm, lower than the surface tension of the fluids so as to provide a contact angle between surface layer and fluid of at least 70°, to prevent the fluid from physically contacting the back sheet.

Document WO 2007/10929 A1 discloses an absorbing article or napkin, including biodegradable end and back sheet elements, wherein said absorbing article further comprises, as essential components thereof, plant seeds embedded therein.

Document DE 19 133 U1 discloses an absorbing article which, even if it is a compostable one, exclusively includes plastics materials of which no mutual ratio is disclosed.

Document U.S. Pat. No. 4,944,734 A discloses a biodegradable absorbing construction comprising, as essential components thereof, biodegradability improver grains including an enzyme, in particular cellulase or emicellulase, embedded therein. The outer layers of this construction are constituted by is biodegradable materials such as processed paper or rayon.

### SUMMARY OF THE INVENTION

The aim of the present invention is to provide a single-use or disposable hygienic absorbing construction or napkin overcoming the above mentioned drawbacks of prior absorbing constructions or napkins.

Within the scope of the above aim, a main object of the invention is to provide such an absorbing construction allowing to reduce any environmental impacts, and being made, at least for a main part thereof, or even exclusively, of biodegradable components or materials.

A further important object of the present invention is to provide such an absorbing construction including a novel superabsorbing material, in the form of grains or a powder, of a truly natural nature, which may constitute a useful alternative material not only for other natural superabsorbing materials, as those which are generally used in napkins, but also for synthetic superabsorbing materials, thereby allowing to replace the latter, preferably completely, but also partially, by the novel inventive superabsorbing material.

Yet another object of the present invention is to provide such an absorbing construction meeting the market requirements in the terms of operating efficiency and performance, without harming the user's health.

Yet another object of the present invention is to provide such a construction which, owing to its specifically designed structural features, is very reliable and safe in operation.

According to one aspect of the present invention, the above mentioned aim and objects, as well as yet other objects which will become more apparent hereinafter, are achieved by a sanitary absorbing construction according to claim 1.

### BRIEF DESCRIPTION OF THE DRAWINGS

Further characteristics and advantages of the present invention will become more apparent hereinafter from the following disclosure of a preferred, though not exclusive, embodiment of the invention which is illustrated, by way of an indicative but not limitative example, in the accompanying drawings, where:

FIG. 1 is a top plan view of the sanitary absorbing construction or napkin according to the present invention;

FIG. 2 is a cross-sectional view showing the inventive absorbing construction;

FIGS. 3A to 3E are photos showing testing protocols and means for testing samples for comparing a liquid acquiring rate of three superabsorbing materials, that is the natural material “LYSORB” (which is a commercially available material based on a saccharide compounds), conventionally mainly used for absorbing constructions and also for napkins, the synthetic material SAP AK SA 6O S, conventionally used mainly in napkins, and the novel inventive material, which is a truly natural material derived from industrial wastes of fruit processing, in particular apples, and being preferably used in a powder form, and the use of which in child napkins constitutes a main aspect of the present invention;

FIGS. 4A to 4F are further photos analogous to those of FIGS. 3A to 3E but showing, for comparing purposes, the acquiring rate parameters and time required for absorbing in testing of samples of the above three types of absorbing materials;

FIGS. 5A to 5F are further photos, analogous to the preceding ones, for a comparative testing of the above mentioned three superabsorbing materials for evaluating their liquid absorbing parameters in contact with cellulose pads and for evaluating the sample structure after one day;

FIGS. 7A to 7C are further photos showing the rewetting—moisture releasing properties of the three superabsorbing material samples;

FIGS. 8A and 8B are like photos of a modified absorbing testing performed on a powder or flour material derived from apple processing wastes, said powder having grains of a particle size preferably larger than 100 microns; and

FIGS. 9A to 9C are further photos relating to a rewetting testing performed on the inventive superabsorbing apple powder material.

### DESCRIPTION OF THE PREFERRED EMBODIMENTS

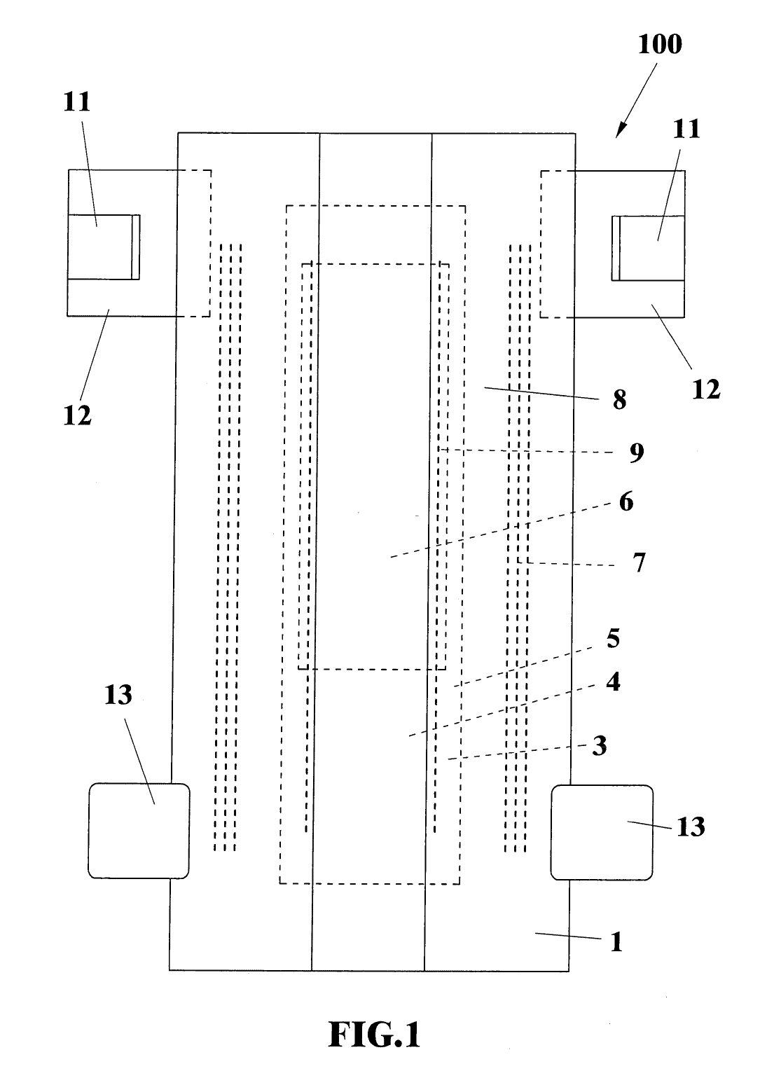

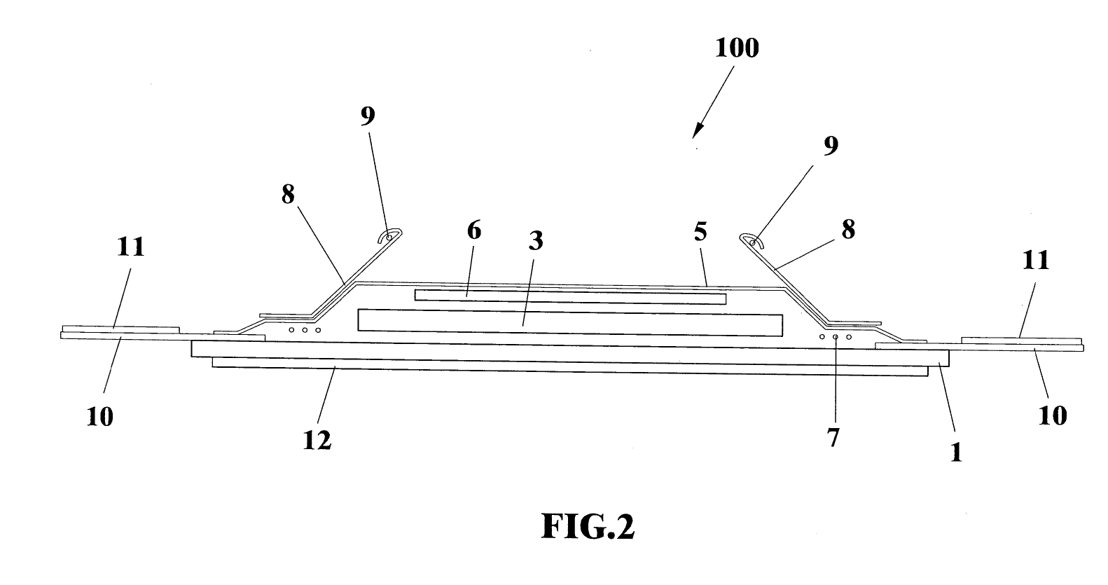

With reference to FIGS. 1 and 2, the sanitary absorbing construction or article according to the present invention, which has been generally indicated by the reference number 100, comprises a barrier layer 1 for contacting the underwear of a child or infant, and a filtering layer 5 for contacting the child body.

Within the above mentioned layers other layers are provided facilitating an inward filtering of liquid materials and restraining the moisture from flowing back toward an outer filtering layer 6.

Said inner layers comprise the absorbing pad proper, that is a layer constituted by two basic elements 3 and 4, such as a cellulose fiber and in general grains made of a superabsorbing material, such as sodium polyacrylate, in which the most part of the liquids is restrained.

At the side portions, through the overall length of the napkin, are provided perspiring barriers 8 for preventing the liquid and solid materials from exiting the side portions.

To the two bottom end portions of the napkin are applied two wings 10, made of an elasticized or resilient materials, with an anchored closure system 11, in turn attached to a front strip 12 whereas, at the two top sides, are further provided front wings 13, aiding a proper locating of the napkin closure means.

All the above mentioned layers are held together by a glue material, which is applied either by a spraying or a contacting method or, alternatively, by other binding systems known in this field, such as a layer thermomelting by ultrasounds.

The article thus made achieves the invention object, owing to its capability of chemically transforming all the materials used into agricultural organic amending materials, suitable for making compost, by a process to be carried out by methods according to public rules and private ruling protocols.

To achieve the above, said absorbing article is made, at least by 90% of its overall weight, of vegetable raw materials, or also mineral materials, such as oil, but each adapted, either per se or jointly with the other article components, to be biodegraded by natural biodegrading cycles of 6-9 months, under a combined effect of atmospheric agents, bacteria, enzymes, moulds, yeasts, which, under given controlled conditions, can be biodegraded even in a biodegrading cycle of 90 days, as provided by the EU 13432 and following rules, and to sanify the pathogen bacteria organic waste materials owing to the temperature of the transforming cycle controlled according to prior methods, which occurs in composting systems because of the organic residues, either of foodstuff or not, as in the case of a napkin, at a temperature higher than 65° C., due to the bacteria natural activity.

Moreover, the subject absorbing article or construction is adapted to be chemically transformed into an organic amending material, independently of the dejections acquired by the article during its use.

The materials suitable for meeting the above requirements are made by using, as a basic raw material, biopolymers, constituting the so-called “bio-based fibers”, that is synthetic biodegradable fibers, the raw materials of which are anyhow of a plant origin, bio-films, which may be both considered as conventional “bio-plastics”.

As a raw material are herein further used high module cellulosic fibers, to be disclosed in a more detailed manner hereinafter, and grains with a high absorbing capability, constituted by a vegetable raw material and so assembled as to form a multilayer combination of half-processed materials such as non-woven fabrics (that is fabrics without warp and weft arrangements) and films (also known as “membranes”).

Contrary to the raw materials used so far in the above articles for forming the filtering and barrier layers, derived from oil derivatives, the novel raw materials for making the present biodegradable articles are polysaccharides mainly derived from plant or vegetable materials, such as starches, flours, cellulose and saccharide derivatives.

As stated, a main aspect of the present invention is that, as a superabsorbing material, is herein used an apple waste powder, derived from industrial processing of apples.

Advantageously, the inventive polysaccharide materials, preferably the apple waste powder, have a thermal hygrosensitivity higher than that of mineral materials, such as oil derived plastics, and, in the absorbing article transforming and assembling steps, require great attention in controlling the air moisture and temperature conditions in the processing systems under any climatic inner and outer conditions with respect to the production environment or room.

The absorbing construction according to the present invention provides many advantages compared with the prior art absorbing articles.

A main advantage of the present invention derives from the environment protection capability.

In fact, conventional child napkins, together with the organic waste material contained therein, represent such a waste mass causing a continuously increasing economic and social problem.

To properly dispose of said waste materials, in particular in highly urbanized areas, it is necessary to improve or enhance said waste materials by recycling at least some components thereof, such as the mixed plastics and cellulose.

Alternatively, said waste materials could be transformed into green energy, by a biogas production and/or a bio-transforming into organic substances such as the so-called “compost”.

Compared with prior methods for selecting and recovering urban waste materials, the existing method for transforming the above waste materials into biogas and compost is the most economical and efficient one among prior to industrial transforming or soil discharging processes, since it does not require any pre-processing steps with related energy consumption and costs, and may be also carried out on mixed organic waste materials.

The present invention greatly reduces the disposal cost while reactivating the starting raw material vital and economic cycle by providing either a biogas, is when the above raw materials are processed in biogas production systems (anaerobic processing), or a compost material, when the above raw materials are processed in a dedicated processing system either as a residue of a biogas production (anaerobic processing) or directly by a waste separation of the urban cycle (aerobic processing).

This amending material is useful for reactivating degraded soils, from which any agricultural activity has been abandoned, and under risk of desertification.

Moreover, said amending material reduces the risk of soil washing away because of a progressive reduction of the biomass in its fertile layer with a consequent loss of porosity and filtering capability, also as a consequence of a heavy use of mineral or synthetic fertilizing materials, in particular in intensive cultivations, and, moreover, it does not destroy environmentally useful raw materials.

With respect to the health protection problem, conventional napkins mainly made of synthetic raw materials or naturally water-repellent materials, require a use of chemical additives to capture and filter away liquids.

Furthermore, prior synthetic polymeric napkins require an application of creams or other chemical elements for reducing friction and contact heat acquiring and entrapping.

On the contrary, the inventive sub-layers for directly contacting the user's skin, being made either by a textile method or a non-woven material method, are constituted by natural and biodegradable raw materials, performing both the above mentioned functions, that is restraining liquids by the perspiring barriers 8, and filtering by the filtering layer 5.

Thus, is herein used a pla (poly lactic acid) fiber, made by a lactic acid polymerizing process and a special cellulosic fiber made by a Lyocell technology, the so-called “Tencel© type Biosoft” method, from the company Lenzing AG, which is a naturally water-repellent material, differently from the known cellulosic fibers which, on the contrary, absorb and hold liquids.

Another preferred material having a high absorbing capability is of the type is disclosed in the Italian patent No. 1402479, disclosing a liquid absorbing barrier, in particular for hydrocarbon liquids.

In both cases, for a proper operation, the method for making the non-woven material which, in the case of the barriers 8, comprises a thermo-bound non-woven material with an even surface and which, in its inner filtering portion 5, also called ADL, comprises a perforated non-woven material, has a primary importance.

The above materials, contacting the skin, provide natural properties, such as softness, are not cytotoxic and cannot accumulate and hold the body heat, differently from the synthetic plastics materials, thereby being very suitable for contacting the human body that has an average temperature lower than 40° C.

Moreover, both the synthetic fiber pla and the synthetic fiber Lyocell, in its so-called Tencel Biosoft form, have an outer structure which is sufficiently even, smooth and compact, contrary to the oil-derived and natural fibers such as cotton, or other synthetic fibers such as Rayon and Lyocell in the conventional form, which are very uneven and do not favor a rubbing friction reduction, contrary to the inventive raw materials.

The outer barrier may also be made of a membrane which, differently from oil-derived synthetic membranes, is naturally permeable to water steam, thereby it may be defined as a perspiring membrane, while providing a suitable barrier against migrating liquids.

To achieve the above results, the component elements are so made that the overall absorbing article contains a rate of biodegradable raw material, of the above disclosed type, not lower than 90%, in the form of a fiber, membrane (film) and grains of vegetable or plant, cellulose or biopolymer origins and also of a to synthetic nature, that is derived from oil, such as aliphatic polyester, being therefore biodegradable.

Each of the above components is biodegradable by 90% of its mass, within a cycle of 3 months, in composting system or in systems for providing a controlled processing of organic nature waste materials, with the exception of the elements 9-11, since they are at present constituted by non-biodegradable materials, in the article of manufacture being however less than 10%.

The materials used for making the subject napkin, as shown in the drawings, are preferably as follows.

The element 5, that is the outer filtering layer, is made of a perforated non-woven fabric which is thermo-bound and/or intertwined by high pressure water jets (spunlaced) and being constituted by a mainly (over 90%) or fully biodegradable Lyocell or Tencel Biosoft type fibers.

The element 6, that is the filtering layer (the so-called ADM) used for filtering purposes and for reducing the back moisture (rewetting effect) is made of a non-woven fabric material mainly constituted (over 60%) or 100% constituted of thermo-co-bound pla fibers.

The element 3 is an inner absorbing layer made of a cellulose fluff material (absorbent core) directly coupled on the napkin assembling line to the element 4 of a non-woven fabric material made by an air-laid or the so-called needle punching method, and including a mixture of cellulosic fibers and grains of high absorbing power, as above disclosed, or simply grains dispersed through the cellulosic absorbing fluff.

The element 7 is constituted by mono-filaments of an elastomeric material arranged between the filtering layer 5 and the barrier layer 1, in a number of two both on the right and on the left side of the napkin main body.

The element 1 comprises a biodegradable biopolymer film which, owing to its water steam permeable nature, is also defined as a “perspiring film”.

The element 8, that is the perspiring barriers, comprises two thermo-bound non-woven fabric material strips and/or is made by a spunlacing method, and is mainly (over 90%) or fully constituted by natural hydrophobic fibers, incapable of acquiring liquids through their structure, and in particular the pla and Lyocell fibers in their Tencel Biosoft form, as above disclosed, without additional chemical processings, either directly on the fiber or on the non-woven material as the latter is made.

Said strips are arranged on the right and left sides of the napkin, being glued or thermo-sealed to the napkin main body, or directly on the element 5, and constitute the side barriers for preventing liquids from exiting while restraining therein the organic solid waste material.

The element 9 and the element 7 are used for adjusting the side barrier tension, for adhering to the user's thighs.

The element 10 is constituted by two wings (stretchable belt or closure) likewise spaced from the napkin edge and perpendicular to the napkin main body.

Each said wing is constituted by two non-woven materials bound by thermosealing or glue materials, and mainly (over 80%) or fully constituted by vegetable origin and biodegradable thermosealable (when subjected to a thermal processing) fibers, such as the pla fiber or biodegradable polyester fiber and including one or more strips extending in parallel to the main body of the article and made of a non-biodegradable elastic synthetic material.

The element 11 is constituted by a strip mainly of a paper material, thereon an adhesive glue material is spread.

The element 12 is constituted by a paper strip applied on the element 1 to aid a proper arrangement or locating of the element 11 on the napkin body to allow the latter to be properly worn by the user (frontal tape).

The element 13 is constituted by two front wings arranged, like the element 10, on the left and right sides of the main structure of the napkin, but at the other end portion of the article main body.

Said wings are constituted by a non-woven material made by a thermo-bonding method and/or spunlaced, in a prevailing manner (over 90%) or fully, of a synthetic fiber of a vegetable origin such as the pla fiber and/or Lyocell fiber.

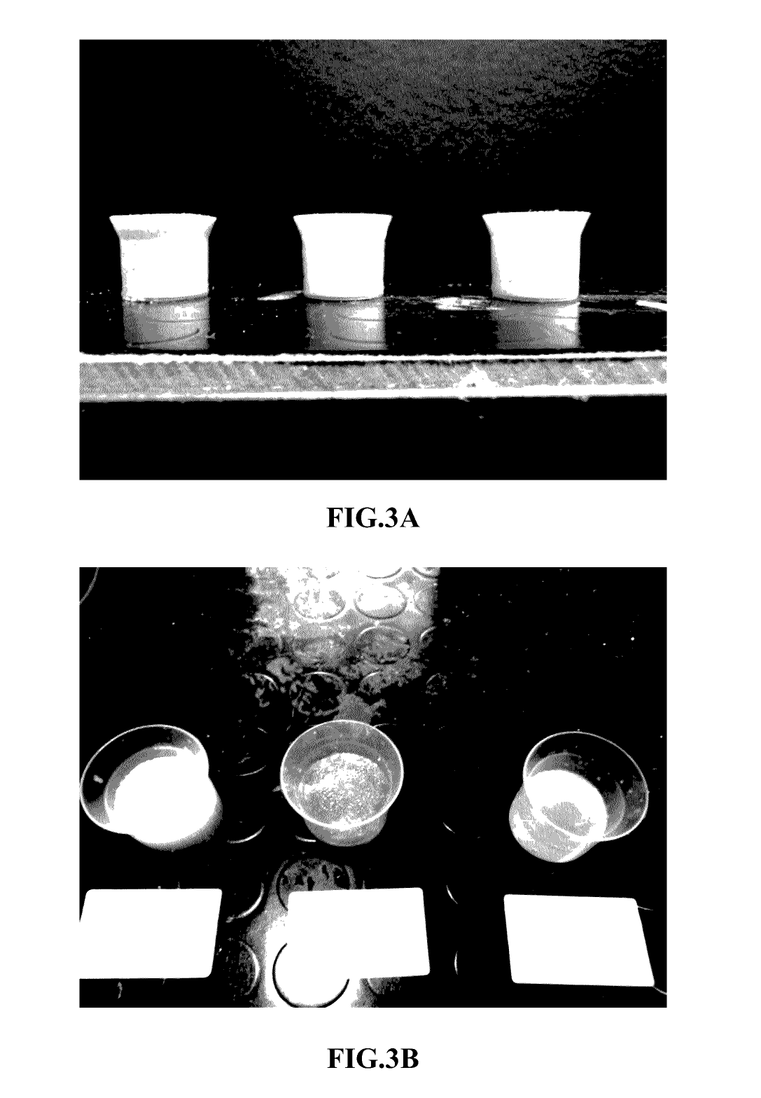

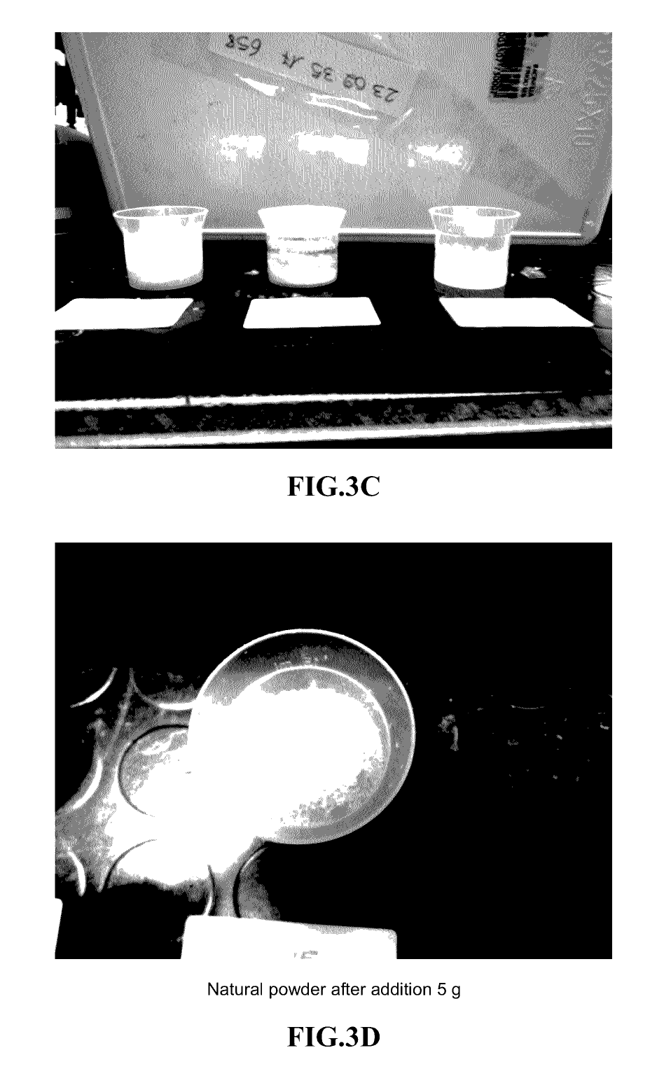

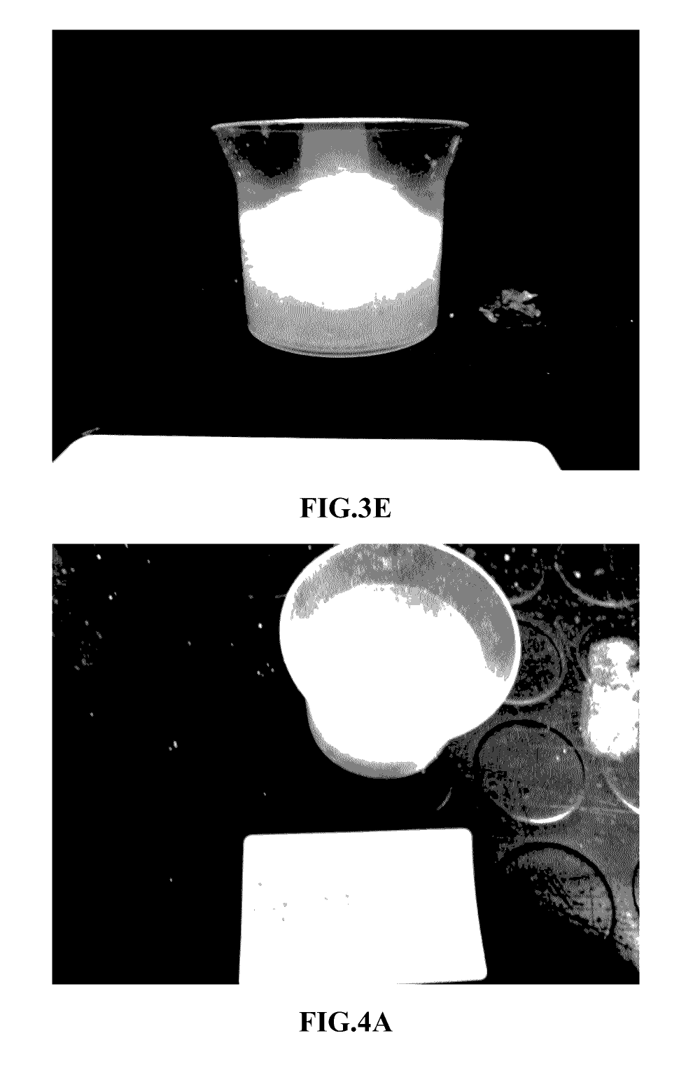

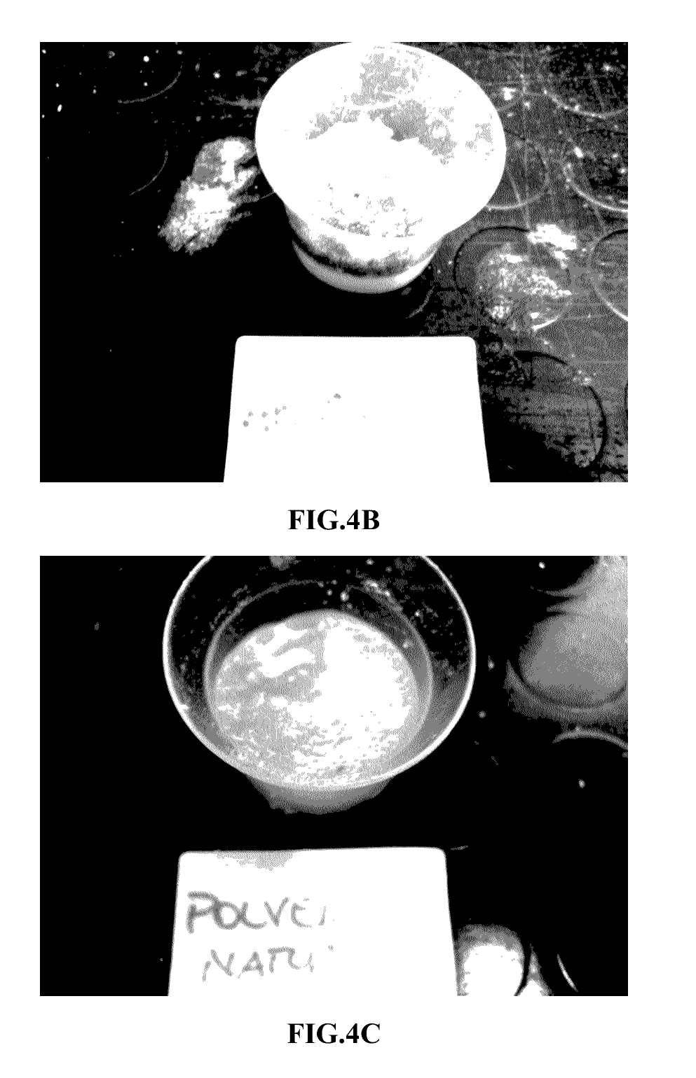

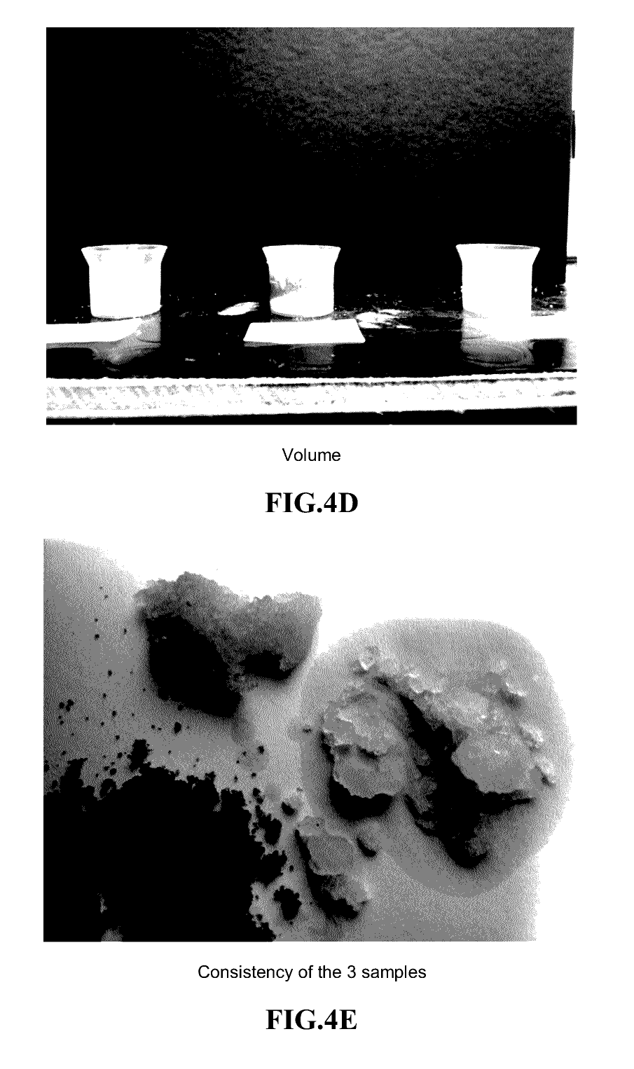

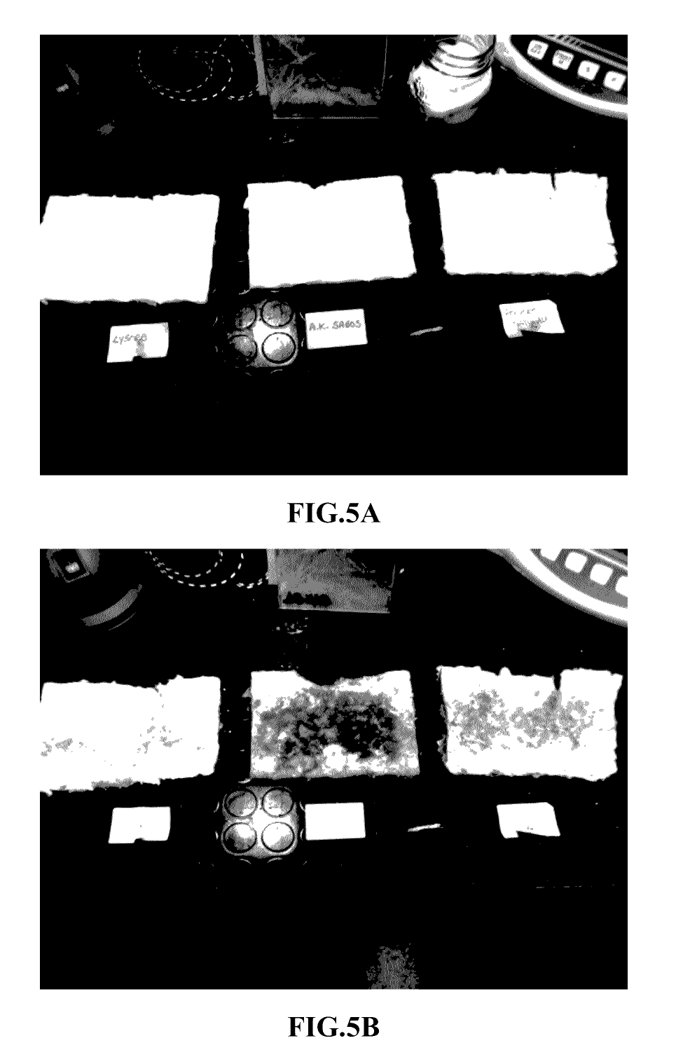

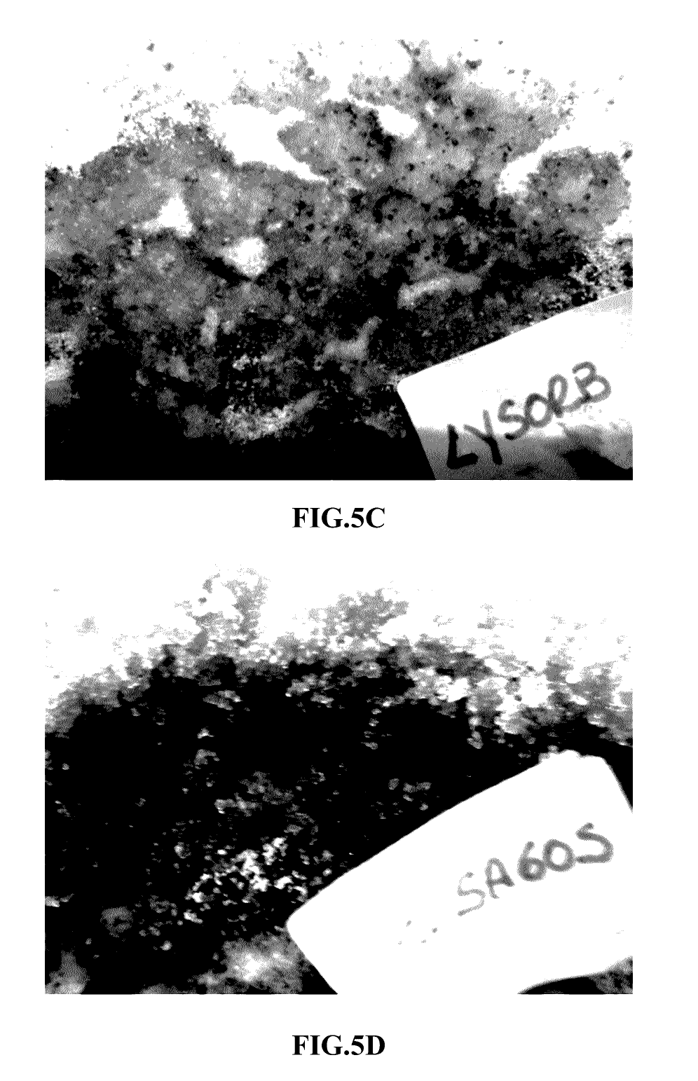

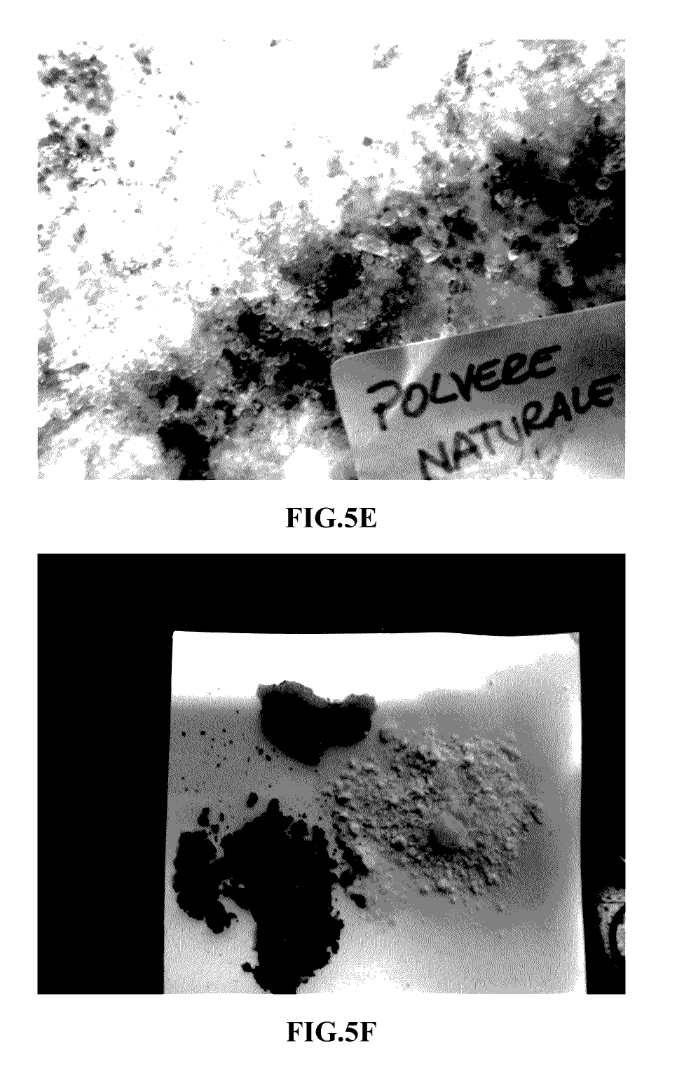

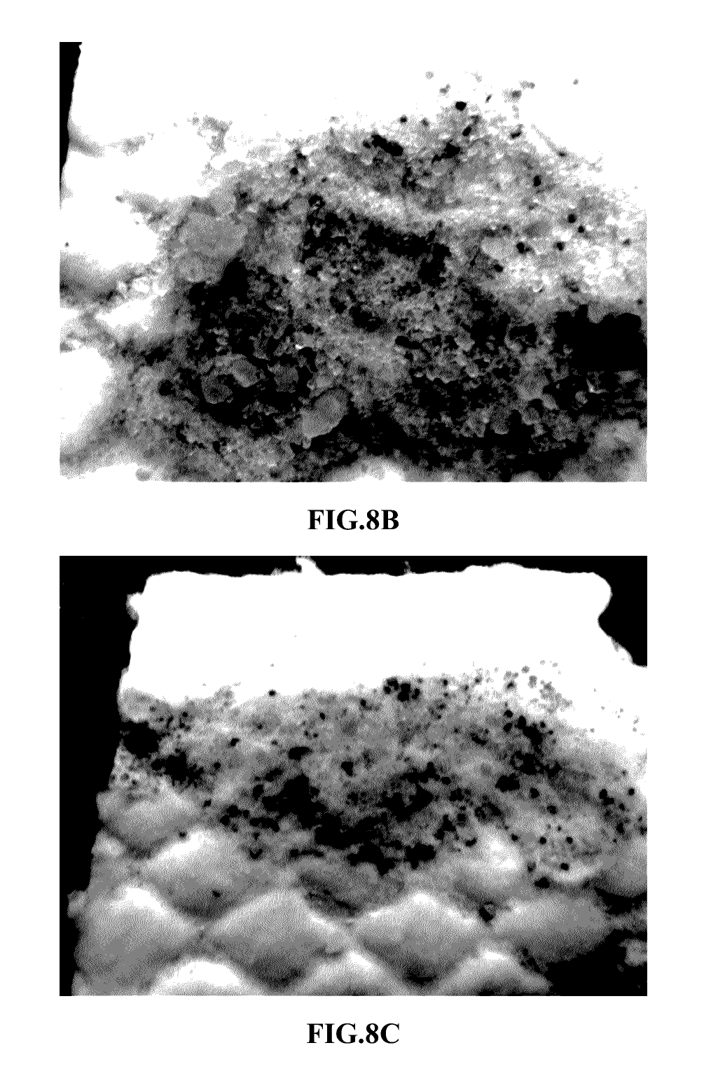

With reference to FIGS. 3A to 3E, 4A to 4E, 5A to 5F, 7A to 7C, 8A and 8B, 9A to 9C, testing procedures are hereinbelow disclosed for testing prior superabsorbing materials, in particular the natural LYSORB material, the synthetic SAP AK SA 60 S material and the apple residue or waste powder constituting a main aspect of the present invention.

Specific Weight as the 3 Different Samples have a Like Volume

| | |
|---|---|
| LYSORB | 15.04 g |
| AK SA 60 S | 17.45 g |
| Powder | 16.76 g |

### 1—Acquiring Rate

After having taken 3 g of each superabsorbent material type and arranged them in a vessel, 5 ml of liquid corresponding to 6.37 g were poured for establishing the acquiring rate.

After 5 minutes
LYSORB acquires in a very slow manner, and absorbs all the liquid AK SA 60 S acquires with a high speed or rate, and not all the sap amount is required (it is known for its high absorbing capability, about 20% higher than that of conventional sap materials)
The natural powder does not absorb all the liquid and it is necessary to add further 5 g to complete the operation

Weight Determined After Liquid Absorption

| | |
|---|---|
| LYSORB | from 3 g to 8.56 g |
| AK SA 60 S | from 3 g to 7.63 g |
| Powder | from 8 g to 12.68 g |

### 2—Absorbing Time

The 3 samples are prearranged in an amount of 3 g for LYSORB, 3 g for AK SA 60 S, 8 g for the powder, 5 ml liquid are poured and the liquid acquiring times are detected.

For LYSORB the liquid is mixed and absorbed in 2 min. and 40 sec.

For AK SA 60 S the liquid is mixed and is absorbed in 1 min and 60 sec.

For the powder the liquid is mixed in 10 sec, but the powder becomes dense in 5 min.

3—Absorbing Test in Contact with the Cellulose Pad

The cellulose pad is divided into 3 sectors, on each sector being spread 3 g is of the different sap materials, covered by a cellulose layer and being then poured 40 ml liquid corresponding to 40.57 g.

| | Dry weight | Wet weight |
|---|---|---|
| LYSORB | 13.04 g | 71.69 g |
| AK SA 60 S | 11.96 g | 90.24 g |
| Powder | 16.60 g | 74.06 g |

After 10 min, the first cellulose layer is removed and the sap material status and structure are verified.

Consistency or firmness of the samples after 1 day: LYSORB and sap remain agglomerated in a jelly form, whereas the powder returns to a powder status.

### 4—Rewetting-Moisture Releasing

The pad is divided into 2 parts or sectors, on the left side are arranged 3 g of Sap Ak Sa 60 S, and on the right side are arranged 8 g of powder. The cellulose layer, tissue, filtering and top sheet are properly arranged.

Into the 2 sectors are poured 15 ml liquid, arranged weights of 4.5 kg for 2 min, arranged dry paper filters previously weighted by a rubber plate, weight of 4.5 kg for 10 min. and the filters are then weighted. The difference between the dry filter weight and wet filter weight determines the release with visible sap.

Rewetting Data

| | dry filter weight | wet filter weight | rewetting |
|---|---|---|---|
| SAP AK SA 60 S | 8.81 g | 11.72 g | 2.91 g |
| Powder | 8.98 g | 10.47 g | 1.49 g |

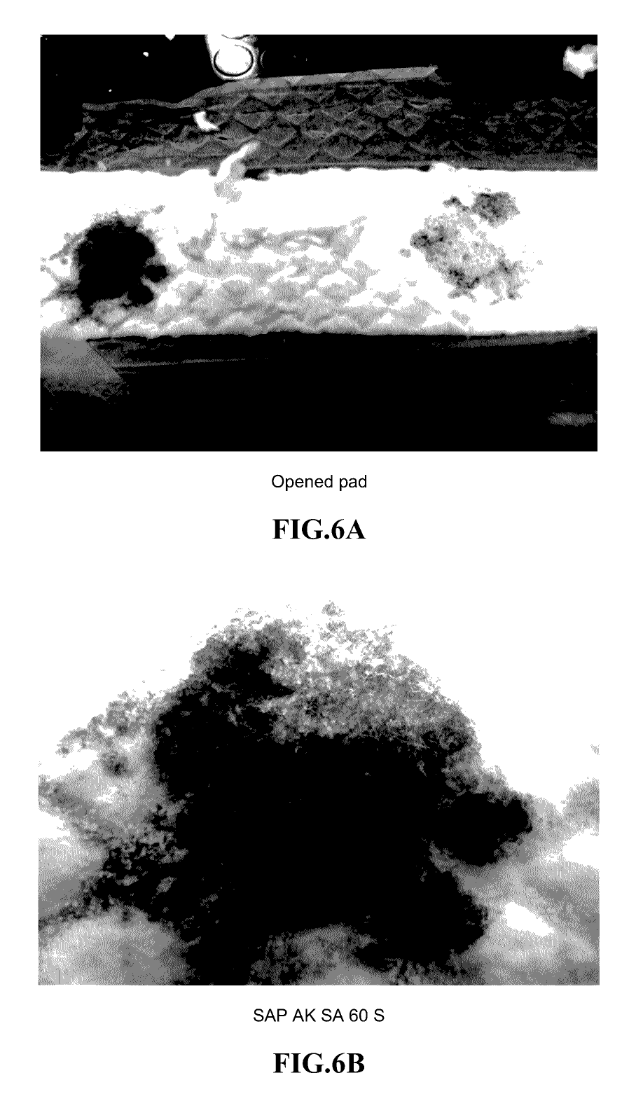

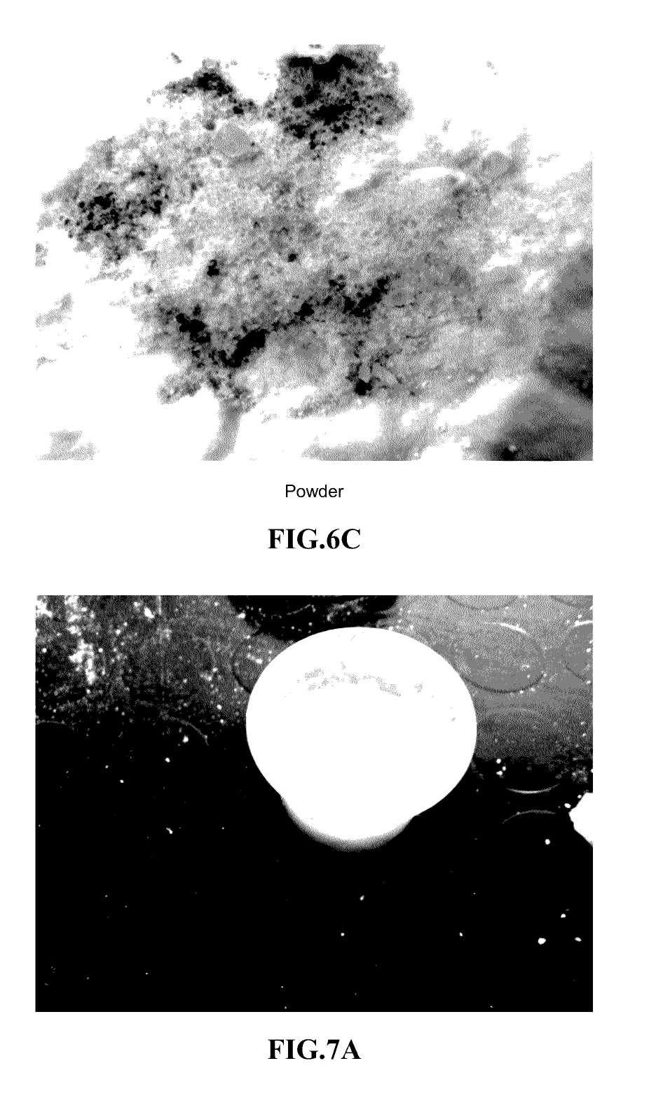

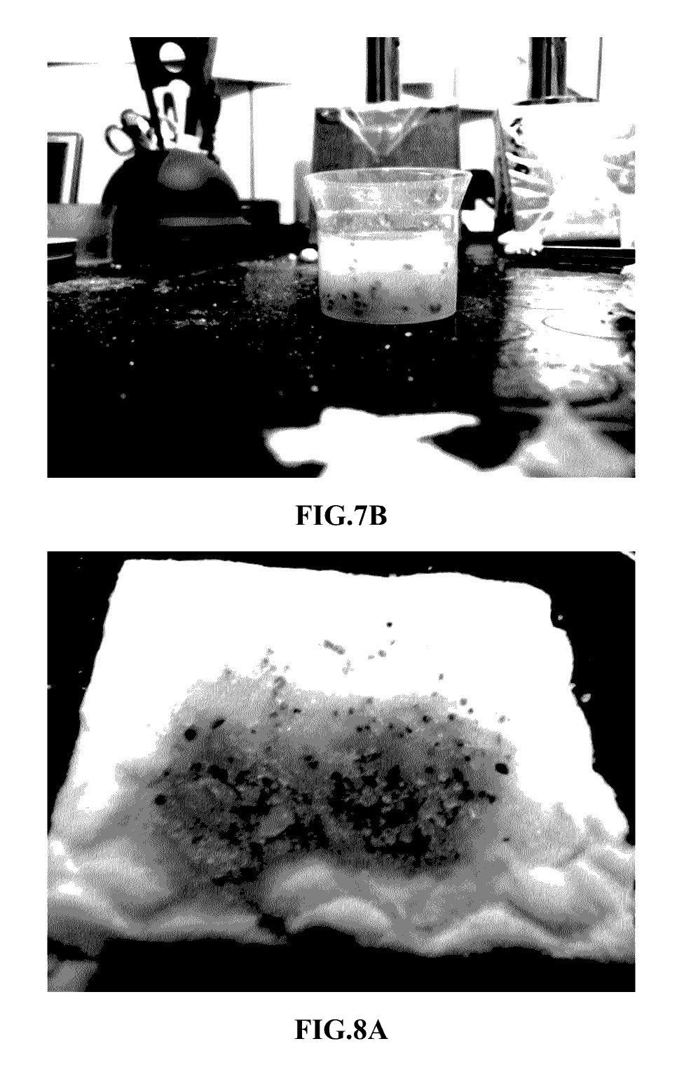

From FIGS. 7A to 7C, it should be apparent that the natural sap LYSORB does not assume after absorption a gel form typical of a normal sap, since its to liquid acquiring rate is, as is known, a very slow one, and this does not assure a full absorption, but favors a dispersion of the liquids through the cellulose pad.

SAP AK SA 60 S provides a perfect absorption, and is swollen as usual.

Unexpectedly, the powder achieves the same result but, while 3 g of the preceding samples have been used, for this powder sample 8 g are used, as in is the preceding test.

However, the end weight of wetted pad is not doubled, even if the powder is very light.

After one day, while normal saps remain agglomerated in a gel form, the powder returns to a powder status.

The rewetting is lower in the sector where the powder is present and this seems to be due to the fact that the liquid is dispersed on the pad to a larger degree and does not concentrate only at a point, since the powder tends to solidify later than the sap.

However, a different situation occurs where the sap is present, since the sap draws the liquid and concentrate it thereon solidifying, thereby preventing any dispersions on the pad.

### 5—Modified-SAP Testing

An absorbing test has been carried out using 8 g modified powder and by pouring 15 ml liquid.

No significant improvement has been found. The powder immediately acquires the liquid but becomes solid in 5 min, as in the previous test.

### 6—Rewetting Test

| dry filter weight | wet filter weight | rewetting |
|---|---|---|
| 8.95 g | 8.99 g | 0.04 g |

In this case, the moisture release on the pad is significantly reduced, since it drops from 1.49 g to 0.04 g upon the modification.

In this connection, please see the sequential photos of the effect on the cellulose pad, clearly showing that the liquid is not dispersed, but is immediately captured and the powder is quickly agglomerated in one minute. Amounts used: 8 g powder, 15 ml liquid.

### Conclusions and Remarks

From the tests carried out on the inventive superabsorbing powder, it should be clear that this truly natural component constitutes a valid alternative to the natural sap LYSORB, which in this case is not suitable for use in napkins since it has a very low liquid acquiring rate, but is more suitable for absorbing constructions, and further to replace the synthetic sap AK SA 60 S conventionally used in making napkins, even if it may involve a larger amount in the making process, or when mixing of the two products.

It should also be pointed out that during the tests a very good absorption as well as rewetting and acquiring rate have been found.

### 7—Test Carried Out for Establishing a Possible Hospital Use

This test has been carried out on samples constituted by portions of biodegradable and synthetic fabrics for establishing their different reaction modes.

20 ml synthetic blood have been poured on absorbing powder which satisfactory reacts to the absorption, without developing excessive smells, which, however, were more present on the synthetic fabric.

The samples have been subjected to a constant temperature of 37° C. for 30 minutes, to simulate the body temperature. A smell has developed in a stronger manner in both fabrics, to be then attenuated during the sample cooling down.

Thus, because of the interesting reactions to this test, the material may be used in hospital aids.

It has been found that the invention fully achieves the intended aim and objects.

In practicing the invention, the materials used, as well as the contingent size and shapes can be any, according to requirements.

1-7. (canceled)

8. A single use sanitary napkin absorbing construction for children, free from contraindications for the health of a child wearing it, not causing skin irritations or abrasions and allergic reactions even for children suffering from dermatitis, psoriasis and other skin diseases, or in a stressed or hardened skin condition, at the end of its useful life, may be transformed, in a truly natural manner, into novel environmentally useful organic substances, for manuring agricultural soils polluted and impoverished by intensive agriculture methods, and for providing gas materials for generating clean energy by controlled natural fermentation and related anaerobic processes, said sanitary absorbing construction comprising textile and plastics sub-layers consisting of at least 90% of raw materials derived from polysaccharides or other polymeric materials of mineral or vegetal origin, all said materials being of a biodegradable nature, from cellulose and derivatives thereof such as cellulose fibers from an agricultural cultivation, which, because of their nature, even combined together and in optimum conditions, are compostable under aerobic conditions according to rule 13432EU, and in which is present, in a form of different size granules, an absorbing material derived from apples, even from industrial apple processing waste, either from a dehydrating or a refining process.

9. An absorbing construction, according to claim 8, characterized in that said raw materials are selected from biopolymers, cellulose fibers and superabsorbing grains, consisting of a vegetal origin raw material and being assembled in a multilayered combination, semi-processed as non-woven fabrics and films or membranes, and polysaccharides mainly derived from vegetable materials such as starches, flours, cellulose derivatives and saccharides, and that one or more sub-layers thereof comprise a vegetal raw material such as dehydrated apple flour or powder or grains.

10. An absorbing construction, according to claim 8, characterized in that said absorbing construction comprises an absorbing pad consisting of two basic elements including an inner absorbing layer of a cellulose fluff material and a non-woven fabric comprising a mixture of cellulosic fibers and highly absorbing grains, or grains dispersed through the absorbing cellulose fluff material.

11. An absorbing construction, according to claim 8, characterized in that said construction comprises perspiring side barriers, consisting of two side non-woven fabric strips, and side front wings; said barriers and wings consisting of a non-woven fabric, of a spunlaced or thermally bound type, made of cellulose fibers, per se or in a mixture with pla fibers.

12. An absorbing construction, according to claim 8, characterized in that said construction comprises a filtering layer applied to the multilayer absorbing pad and consisting of non-woven fabric cellulosic fibers per se and/or in a mixture with pla fibers, said filtering layer being either glued or thermosealed to a biodegradable biopolymer film.

13. An absorbing construction, according to claim 10, characterized in that said absorbing pad comprises a non-woven fabric material, of an air-laid or spunlaced type, and being made of mixture of cellulose fibers and absorbing fibers.

14. An absorbing construction, according to claim 8, characterized in that said construction comprises two wings, of an elasticized material, to which is anchored a closure system comprising a closure strip mainly made of a paper material thereon an adhesive glue is spread, said paper strip being coupled to a front strip; said closure system comprising two layers thermo-bound or glued to one another including one or two film strips containing an elastomeric polymer.

15. A sanitary absorbing construction, according to claim 8, comprising at least the following components: an outer filtering layer made of a perforated non-woven fabric which is thermo-bound and/or intertwined by high pressure water jets and constituted by over 90% or fully biodegradable fibers, a rewetting reducing further filtering layer made of a non-woven fabric material constituted by over 60% or 100% constituted of thermo-co-bound pla fibers, an inner absorbing layer made of a cellulose fluff material absorbent core directly coupled to a non-woven air-laid or needle punched fabric material element including a mixture of cellulosic fibers and grains of high absorbing power, or grains dispersed through the cellulosic absorbing fluff material, a further element constituted by mono-filaments of an elastomeric material arranged between the outer filtering layer and a barrier layer, in a number of two both on a right and on a left side of a napkin main body, said barrier layer comprising a biodegradable biopolymer perspiring film, a further perspiring barrier element comprising two thermo-bound non-woven spunlaced fabric material strips constituted by over 90% or fully constituted by natural hydrophobic fibers, said strips being arranged on the right and left sides of the napkin, being glued or thermo-sealed to the napkin main body, that is directly on said outer filtering layer, and for preventing liquids from exiting while restraining organic solid refuse or waste material, further element for adjusting a side tension, two stretchable belt or closure wings spaced from a napkin edge and perpendicular to the napkin main body, each said wing being constituted by two non-woven materials bound by thermosealing or glue materials, and constituted by over 80% or fully by vegetable and biodegradable thermosealable fibers, comprising pla fibers or biodegradable polyester fibers and including one or more strips extending in parallel to the napkin main body, a first paper strip, thereon an adhesive glue material is spread, a second paper strip applied on said barrier layer to aid a proper arrangement of said first paper strip on the napkin body to allow the latter to be properly worn, and two front wings arranged on the left and right sides of the napkin, but at the other end portion of the article main body, said front wings being constituted by a non-woven thermally or spunlaced material made by over 90%, or fully, of a synthetic fiber or a vegetable fiber.
